(19) United States
(12) Patent Application Publication
(10) Pub. No.: US 2020/0345286 A1
(43) Pub. Date: Nov. 5, 2020

(54) CASSETTE DEVICE FOR QUICK TEST OF DIAGNOSIS, METHOD FOR DETECTING A LIGAND IN A BIOLOGICAL SAMPLE AND KIT

(71) Applicant: ORANGELIFE COMÉRCIO E INDÚSTRIA LTDA - ME, Rio de Janeiro (BR)

(72) Inventor: Marco Collovati, Rio de Janeiro (BR)

(21) Appl. No.: 16/536,090

(22) Filed: Aug. 8, 2019

(30) Foreign Application Priority Data
May 3, 2019 (BR) 102019 009125-8

(51) Int. Cl. A61B 5/15; G01N 33/558; A61B 5/157

(52) U.S. Cl. CPC A61B 5/150022; A61B 5/15186; A61B 5/157; G01N 33/558

(57) ABSTRACT

A cassette device for rapid diagnostic test in a single step. More specifically, an immunochromatographic rapid test for identifying antibodies present in a biological sample, such as human blood, is disclosed. The device has a lower cassette (1) and an upper cassette (7) containing a capillary-tube-shaped structure (8). The capillary-shaped structure holds the sample amount needed for the rapid test, preventing waste and sample dilution. Lower (1) and upper (7) cassettes are attached by a movable cover (9), which when pressed causes the capillary-tube-shaped structure closure (8), and a disruption of a capsule (5) containing buffer solution by pressing thereof using a spear-shaped structure (6). Disruption of the capsule (5) by this mechanism allows the buffer solution to be poured in a controlled manner over an absorbent pad (3) located on a test strip (2) in a point anterior the point receiving the sample (10).

### FIELD OF THE INVENTION

The present invention belongs to the field of integral devices, and methods for detecting a ligand in a biological sample.

Generally, the present invention refers to tests by methods involving the formation of biospecific bonds of ligands. More specifically, the present invention aims to measure immunologics for the purpose of diagnosis. In other words, the present invention discloses a device for rapid detection of an antigen linked to an antibody for the purpose of diagnosis.

The present invention therefore refers to a rapid test device for verifying the immune response of an individual. More specifically, the present invention refers to a lateral flow immunoassay device, or still, an immunochromatographic in vitro rapid diagnostic test device. Still more specifically, the present invention relates to a device for immunologic test for detecting a ligand present in a biological sample, such as blood.

Still it is disclosed a kit for rapid test of biological samples, such as blood, for diagnosis purpose.

### BACKGROUND

There are a number of ways of testing for disease status and verifying an individual's health situation, but in general, such tests or trials may be classified into two main groups: those that are intended to be run by the physician or other practitioner in the health area in the laboratory, clinic or hospital environment, and those that can be run by the patient himself.

Rapid diagnostic tests play an important and growing role in patient care worldwide: they can be administered by both physicians and other health practitioners in laboratories, hospitals, medical clinics in urban or rural areas (at the point of care), as well as by the patient himself aiming at a better control of certain chronical diseases (e.g. such as diabetes), or for determining certain clinical status, as for example the determination of pregnancy, as in the case of the test for the qualitative determination of human chorionic gonadotrophin—hCG.

Many types of rapid diagnostic tests are commercially available and are intended to detect the presence of substances (ligands) in biological fluids such as blood, urine, saliva, etc. Non-limiting examples of similar rapid test devices commercially available are, for example, PSA test (for prostate cancer detection), cardiac troponin test (myocardial infarction indicator), test for detection of Helicobacter pylori (indicative of (gastric ulcer), test for faecal occult blood detection (possible colorectal cancer indicator), test for detection of influenza antibodies A+B (H1N1), dengue (NS1—IgG and IgM antibodies), HCV (type C hepatitis), HBsAg (type B hepatitis), HIV (type 1, type 2 and subtype 0), to name a few.

Lateral flow immunoassays, or rapid tests, are those whose running, reading and interpretation of the result are done within a maximum of 30 minutes, without the need for laboratory structure or additional reagents. They are primarily recommended for face-to-face testing and can be done with a venous puncture or a digital pulp sample, with oral fluid samples or even with serum or plasma samples.

Rapid diagnostic test, as disclosed in the present invention, has gained widespread popularity because of its simplicity, its ability to provide accurate information in a short time, in addition to the possibility of testing more than one analyte in a single (or a few) step(s). Generally, it is enough simply adding the sample to the device, and in a few minutes an accurate result may be got.

Advantageously, rapid test devices have a shelf life of 1 to 2 years, without the need for refrigeration. The ease in handling and interpreting the results makes it possible for them to be used by personnel with little or no specific training, such as the patient himself.

A portion of the currently available rapid tests involves a receptor, for example, an antibody, which is specific for a ligand or antigen, and means for detecting its presence. Rapid tests should be specific and sensitive, and their specificity and sensitivity vary depending on the type of test.

Rapid tests may be quantitative or qualitative. In the first case, the amount of a given antibody present in the biological sample (the patient's blood) and its relationship to a particular status or diagnosis is determined, as is the case in certain pregnancy tests with an indicator of number of weeks, a direct amount of beta-chorionic hormone present in the woman's blood (or urine) sample.

Although quantitative rapid tests are capable of running quantitative determinations of the reaction product, most of the time only qualitative determination is required (qualitative test), in which the mere presence or absence of a ligand (which reflects the presence or absence of product of the ligand-receptor reaction) should be detected. As a result, this type of test usually exhibits an observable indicator of the color change of a particular marker, or the appearance of a particular marker at the end of the reaction. Examples of qualitative rapid tests for blood typing, determination of pregnancy and presence of HIV can be easily found on the market.

As reported above, specificity and sensitivity are highly desirable characteristics for this type of assay. However, minimal changes in the reaction components may reflect on the quality and reliability of the result. In other words, the lack of experience of the applicator/final user may generate false-positive or -negative results that strongly impact the treatment of the patient. Incorrect handling, excess of biological sample (or insufficient sample amount), as well as change in reagent volume (and its concentration) are only a few points that may reflect a change in the result obtained.

Many commercially available devices seek to solve the major problems related to the use by the patient himself or other healthcare practitioners. The vast majority of devices, however, has multiple steps, reagents, components and complicated instructions. Some devices seek to establish procedures in a few steps, providing all the material necessary for their execution by the end user. However, handling of this material by untrained personnel can negatively affect the result. Other devices available in the market seek to simplify insertion of the sample into the device, collection of the blood sample, or both. However, even then, the lack of user training often prevents the correct amount of blood sample from being placed in the device, which can also change the result obtained.

In order to overcome these and other disadvantages of the commercially available devices, the inventors have developed the rapid diagnostic test device of the invention, which aims at simplifying the insertion of the patient's blood sample so that only the amount needed for the reaction can be used, preventing that excessive or insufficient amounts of the blood sample come into contact with the other components of the reaction. Still advantageously, the inventors have developed a methodology wherein the reagents required for the reaction are poured in a controlled manner in a single step, in a position forward the sample, promoting at first a small incubation of the antibodies or antigens present in the sample with the buffer solution, and thereafter, at a later time, washing the test strip, removing all components of the sample which did not bind to the test strip or whose binding is non-specific, preventing problems related to inappropriate manipulation and amounts of reagents.

The international patent document No. WO2015/075677, incorporated herein by reference in its entirety, discloses a rapid test device which includes a reservoir for buffer or other test fluid, which can be selectively released by the user in a selective manner through a attenuator controlled by the user in a supplied vessel. The attenuator is responsible for promoting fluid contact with the test material in a controlled manner. The document No. WO2015/075677 also discloses a method for rapid test wherein the user adds the biological sample to the test component operated by the attenuator, releases the test fluid from the reservoir to the control vessel and releases the contents of the control vessel over the test material in a controlled manner. While such a document provides for controlled pouring of the buffer solution (or other test fluid), the document does not disclose or suggest mechanisms for the control of the sample volume by the user.

The American patent document No. US2017/001192, incorporated herein by reference in its entirety, discloses a similar device, wherein the control vessel is absent. The American patent document No. US2017/001192 discloses an integrated rapid test device including a test component, a plurality of reservoirs adapted to contain a test fluid in the form of a sachet and an attenuator responsible for the controlled release of the test fluid into the test component inserted into a support structure. The device further contains a vessel for receiving the biological sample so that the sample can be brought into contact with the test component. Although this document states that the sachet is disrupted by the attenuator in a controlled manner at a specific site, disruption thereof may potentially extrapolate the limits provided for receiving the test solution and cause the test fluid to be driven in other directions, diminishing the performance of the device. The document No. US2017/001192 also does not disclose or suggest means of controlling the volume of sample added.

The international patent application document No. WO2018/085878, herein incorporated by reference in its entirety, discloses an integrated package for delivery of a test fluid and a process for forming such an integrated package. The document No. WO2018/085878 further discloses a process for forming a liquid filled reservoir, with a frangible seal formed by heating which, when disrupted by an operator, releases its contents into a liquid delivering vessel, such as a test fluid, in a controlled manner in a test component, such as a biological sample (fresh blood, plasma or serum, saliva or urine). However, the document No. WO2018/085878, does not disclose or suggest mechanisms to control the sample volume to be tested.

The international patent application No. WO2019/071323, incorporated herein by reference in its entirety, discloses an integrated test unit including a blood receiving recess, a test component, a reservoir containing a test fluid, wherein the blood receiving device operates by actively delivering the blood sample to the test component by activating an attenuator, and promotes the release of the test fluid into the reservoir so as to contact the test component. The blood receiving recess disclosed by the international patent application No. WO2019/071323 is adapted to directly receive the blood sample from a user. Such a recess includes a channel which, when operated, is progressively filled with blood in such a way that the operator can visualize when the recess is completely filled with blood, and verify that sufficient volume of sample has been collected for running the diagnostic test. Although the international patent application No. WO2019/071323 discloses mechanisms for controlling the sample volume, the possibility of using a large volume of blood sample makes the device infeasible for use by an untrained individual. In addition, the disclosed device contains a lancet for perforation of membranes (such as the skin) and collection of blood sample.

The American patent application No. US2018/0272338, herein incorporated by reference in its entirety, discloses a fluid collection unit comprised of a vessel for the passive collection of a fluid sample, such as blood or saliva, the inserted sample goes through a path through the interior of the device, to address the fluid sample from the collection vessel to the opposite end of the unit. The sample then reaches a region which contains a concavity, wherein the separation of solid elements from the sample (as cellular components) occurs allowing for the passage of only the liquid portion of the sample to the most distal portion of the device, wherein the reaction of the sample components with the ligand occurs. No buffer or other test solution is used. The user can view the reaction product through the transparent body of the device. However, despite the possibility of using the device disclosed in document No. US2018/0272338 with blood samples, the document is described for use with oral sample, such as saliva. The device disclosed in the document No. US2018/0272338 also is able to use a large volume of sample (up to about 200 microliters), which can be a disadvantage when used with a blood sample collected by the user.

The U.S. Pat. No. 9,476,875 discloses a lateral flow test device for use in a liquid solution which includes a housing that houses a plurality of adsorbent strips and that at least one strip has a location for receiving the solution. Said housing further has an aperture which is located above the receiving vessel of the solution, aligned therewith. A sealed vessel containing a liquid solution is further provided, the enclosed vessel being disposed in alignment with said aperture and coupled to said housing. Additionally, a reservoir opener is provided coupled to said housing. The opener is arranged to rotate from a first position in which the opener is not in contact with the sealed reservoir to a second position in which the opener grinds the seal of the sealed reservoir to effect the release of the solution from the reservoir to the interior of the aperture and to the adsorbent strip contained in the housing. The test device disclosed by the U.S. Pat. No. 9,476,875 therefore provides a series of components which should be handled by the user, which may lead to potential error, affecting the desired test result.

The device disclosed in the U.S. Pat. No. 9,885,710, hereby incorporated by reference in its entirety, has a system containing more than one test absorbent strip, overlaid one another, and includes a double marking: the reaction product of the first reaction is reagent for the second reaction located over the second adsorbent test strip, providing an indirect method for the detection of the analyte of interest, which may increase the chance of error, and lead to an altered result.

The American patent application No. US2018/0164295, herein incorporated by reference in its entirety, discloses a secure assay device providing a test or assay device that provides at least one result, comprising at least one surface exhibiting critical change, such as optical change, in response to at least one target particle, at least one marker, or a combination thereof; and at least one multilayer coating which at least partially covers the assay membrane, the assay device, or a combination thereof. Said multi-layer coating blocks or prevents the user from viewing the critical change or result. A safe reader and in vitro test method for use with the safe assay device disclosed by document No. US2018/0164295 are also disclosed.

The U.S. Pat. No. 7,959,877, herein incorporated by reference in its entirety, discloses a device for collecting and analyzing a fluid sample that is simple to manufacture, assemble and handle (by the user), and which can be safely used in predetermined volumes (such as 10 microliters) of biological samples, such as blood, serum, saliva or urine. Said device comprises a cylinder defining a chamber containing in its interior an analytical test medium and having a narrow path at one end. The device further comprises an absorbent pad wick to pick-up the sample extending from the interior of the chamber and passes through the narrow path until it comes in contact with the external environment, in addition to a hydrophobic splash filter that prevents excess sample or buffer from reaching the interior of the camera. The document U.S. Pat. No. 7,959,877 also discloses a biological sampling kit containing the device of the invention and additionally a buffer solution in a separate vessel protected by a penetrable sheet.

The invention disclosed by the U.S. Pat. No. 7,959,877 has a complex operation: after collection of the biological sample by any means of the art, the end containing narrow path of the device should be abutted on the blood sample so that it is absorbed by the absorbent pad wick contained in its interior, or, alternatively, the sample should be inserted with the aid of a pipette; the entire end containing the narrow path of the device where the sample is inserted should be hermetically introduced into the separate vessel containing the buffer solution, disrupting the penetrable sheet so that the buffer solution reaches the interior of the device and goes through the same path as the sample to the reaction site inside the chamber. Thus, although the device disclosed in the document U.S. Pat. No. 7,959,877 potentially utilizes a reduced amount of sample and prevents excessive amounts of test solution (buffer solution) from reaching the interior of the chamber and the test site, the mechanism utilized by the device allows for the mixture of the buffer solution with the blood sample into the device cone, causing the sample to be diluted with the buffer. Such dilution is prevented by the device of the invention disclosed herewith because of the position in which the buffer solution is poured into the absorbent pad (prior to the sample). Such positioning is advantageous as it allows for a small incubation of the sample with the buffer solution at first, and the washing of the unreacted components of the sample and removal of non-specific binding at a second time. Additionally, the kit disclosed by the document U.S. Pat. No. 7,959,877 has multiple components and excessive manipulation by the user which may influence the effectiveness and sensitivity of the final result.

Since the prior art documents do not disclose or suggest a simple device that offers a suitable solution to the problems raised, the inventors have reached in the invention disclosed herewith where a device for running rapid test for immunochromatographic diagnosis in vitro set in a single step in a safe and efficient manner utilizing the exact amount of blood sample needed, where the buffer solution to the reaction is poured in a controlled manner in a position prior to the sample, allowing the sample to be firstly incubated with the solution buffer and washing the unreacted components of the sample, as well as removing non-specific bonds, at a second moment in an advantageous manner.

### SUMMARY OF THE INVENTION

In one embodiment, the present invention discloses a device for rapid diagnostic test comprising:

a lower cassette (1) containing recesses where a test strip (2) containing one or more ligands and a positive control of a reaction is positioned; a first absorbing pad (3) positioned over the test strip, upstream thereof; a second absorbing pad positioned over the test strip (4), downstream thereof; a capsule (5) containing buffer upstream the test strip, and a spear-shaped structure (6) located in a position anterior the capsule (5),

an upper cassette (7) having a capillary-tube-shaped structure (8) to be filled with a blood sample, located over the first absorbing pad (3) upstream, and that is fitted to the lower cassette (1) forming an assembly containing an interior cavity isolated from external environment, where the rapid test reaction occurs,

a compressible, movable cover (9) which is connected to the lower cassette and the upper cassette upstream of the capillary-shaped structure, promoting closure of the assembly,

wherein

the compression of the movable cover (9) presses the spear-shaped structure (6) over the capsule (5) containing buffer in order to disrupt said capsule and pouring the buffer solution onto the first absorbent pad (3) located upstream the test strip, wherein the buffer solution goes through the entire test strip (2) until it is absorbed by the second absorbent pad (4) located downstream of the test strip (2),

and, wherein

the compression of the movable cover (9) promotes the closure of the capillary-tube-shaped structure (8).

In an embodiment of the invention, capsule disruption (5) containing buffer occurs by compressing the movable cover (9) over the spear-shaped structure (6), causing the buffer contained in the capsule (5) to be poured in a controlled manner over the absorbing pad (3) located upstream the test strip (2), in region anterior of the region receiving the blood sample (10) from the capillary-tube-shaped structure (8).

In an embodiment of the invention, the buffer displacing in the first absorbing pad (3) and over the test strip (2) pushes the components of the blood sample throughout the test strip (2) in such a way that the assembly formed by the buffer and the components of the blood goes through the entire test strip (2), passing through the regions containing one or more of a sample ligand and a positive control ligand of reaction, until it achieves the absorbing pad (4) downstream the test strip.

In an embodiment of the invention, a capillary-tube-shaped structure (8) is located over the first absorbent pad (3), and in direct contact with the first absorbent pad (3), in a position (10) posterior to the capsule containing buffer (5) and anterior to the portion of the test strip containing one or more than one ligand of a sample, and a positive control ligand for the reaction.

In an embodiment of the invention, the blood sample is inserted to the capillary-tube-shaped structure (8), by microfluidics, such that, after being completely filled, the sample achieves the first absorbing pad (3), and is immediately transferred thereto, causing the capillary-tube-shaped structure (8) to be emptied (8).

In an embodiment of the invention, the capillary-tube-shaped structure (8) is able to hold the exact needed sample amount to run the rapid test of the device of the invention.

In an embodiment of the invention, after transferring the blood sample of the capillary-tube-shaped structure (8) to the first absorbing pad (3) the capillary-tube-shaped structure (8) emptying occurs, preventing transferring additional blood sample volume.

In a preferred embodiment of the invention, the test strip is a nitrocellulose membrane strip.

In an embodiment of the invention, the lower cassette (1) and upper cassette (7) bodies of the device have a transparent material, allowing the user to see the blood sample and the buffer inside the device, from the sample entry to its absorption by the absorbing pad (4) when the reaction ends.

In an embodiment of the invention, the movable cover (9) has an upper cross bar (11) that, when the movable cover (9) is compressed, it is located over the capillary-tube-shaped structure (8) causing the capillary-tube-shaped structure closure (8) and the isolation of the inner portion of the device of external environment.

In an embodiment of the invention, the one or more ligand of a sample located in the test strip (2) corresponds to one or more antigens fixed to the test strip (2) in a portion posterior the region receiving the blood sample (10) from the capillary-tube-shaped structure (8).

In an embodiment of the invention, one or more antigens present in the test strip are complementary to one or more antibodies present in the capillary blood sample.

In another embodiment of the invention, a method for identifying one or more antigens in a biological sample is provided comprising the following steps:

collecting a biological sample, as a capillary blood sample;

filling the capillary-tube-shaped structure (8) of the device, in its entirety with the needed amount of the blood sample collected;

pressing the movable cover (9) of the device, so as to cause capillary-tube-shaped structure closure (8) and capsule compression (5) containing buffer solution, that is poured upstream the biological sample;

waiting for the buffer solution to go through entire test strip (2) until it is absorbed by the absorbing pad positioned over the test strip (4), and downstream thereof; and

verifying the formation of one or more conjugates among the one or more antigens present in the test strip (2), and one or more antibodies present in the biological sample.

In an embodiment of the invention, the sample is a capillary blood, venous blood, tissue fluid sample, or a mixture thereof. In a preferred embodiment of the invention, the biological sample is capillary blood.

In a preferred embodiment of the invention, the method for identifying one or more antigens in a biological sample utilizes a device of the invention.

In another preferred embodiment of the invention, a rapid diagnostic test kit comprising the device of the invention and instructions for use is disclosed.

### Object of the Invention

It is an object of the present invention to provide a cassette device for rapid, simple, efficient and single-step diagnostic test.

More specifically, it is an object of the invention to provide an immunochromatography rapid test for identifying antibodies present in a biological sample, such as human blood in which the blood sample is directly transferred from the patient finger, without the aid of a collector (such as pipettes, microtubes, or other mechanism of transferring biological material known in the art), to the capillary-tube-shaped structure that automatically transfers through microfluidics, utilizing the capillary force, the collected blood to the membrane of the rapid test device in an efficient manner.

It is further an object of the invention to provide buffer solution in a controlled manner for the rapid diagnostic test reaction by avoiding overloading the sample with excess reagents or boosting the sample by uncontrolled pouring of the buffer solution.

The buffer solution poured in a controlled manner onto the first absorbent pad will start the run along the entire length of the nitrocellulose test strip pushing upstream the antibodies or antigens present in the blood sample which will be complexed to the antigens or antibodies fixed on the nitrocellulose test strip.

It is further an object of the present invention to incubate the blood sample with the buffer solution facilitating the migration of the sample onto the test strip and the accuracy of the result obtained. It is a further object of the invention to wash the membrane during passage of the buffer solution through the test region, thereby removing any possible non-specific binding, ensuring the quality and reproducibility of the result obtained with the rapid diagnostic test device of the invention.

It is a further object of the present invention to advantageously promote the closure of the system, preventing possible contamination. Thus, the compression of the movable cover promoted by the user, in addition to initiate the reaction, it isolates the interior of the device.

It is further an object of the present invention to observe the reaction in real time from the time of blood collection by the capillary-tube-shaped structure until the unreacted mixture achieve the second absorbent pad. It is therefore an object of the invention to provide a rapid diagnostic test device in fully transparent material so as to allow the rapid diagnostic test to be viewed in real time.

Advantageously, and unlike conventional devices, the sample is collected and tested in a single action, as discussed above and following.

### BRIEF DESCRIPTION OF THE INVENTION

FIG. 1 shows a deconstructed view of the rapid diagnostic test cassette device of the invention, all of its components being represented, namely: a lower cassette (1), test strip (2), a first absorbent pad (3), a second absorbent pad (4), a buffer solution containing capsule (5), a spear-shaped structure (6) an upper cassette (7), a capillary-tube-shaped structure (8), a movable cover (9), a region that receives the blood sample (10) and an upper cross bar (11).

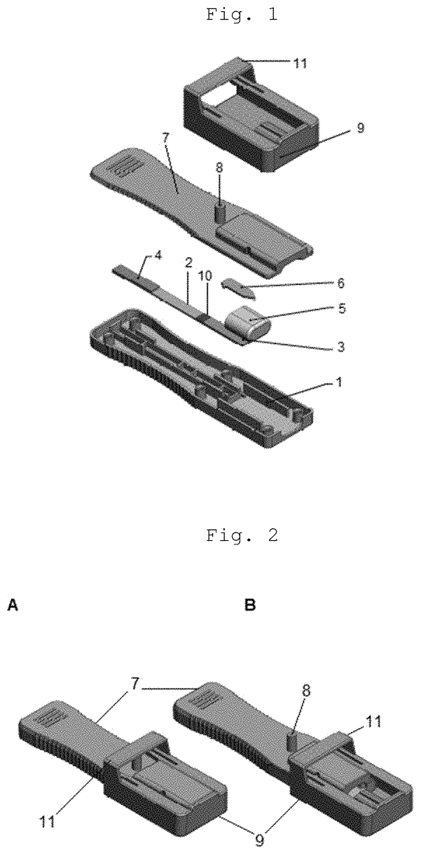

FIG. 2 shows a rapid diagnostic test cassette device of the invention assembled in two views. The panel A discloses a device wherein the movable cover (9) has been compressed so as to promote the closure of the capillary-tube-shaped structure (8) (closed mode) and the disruption of the capsule containing the buffer solution (5) in the interior of the device (not shown). The panel B discloses a device wherein the movable cover (9) is decompressed, that is, the device shows the capillary-tube-shaped structure (8) unobstructed (open mode).

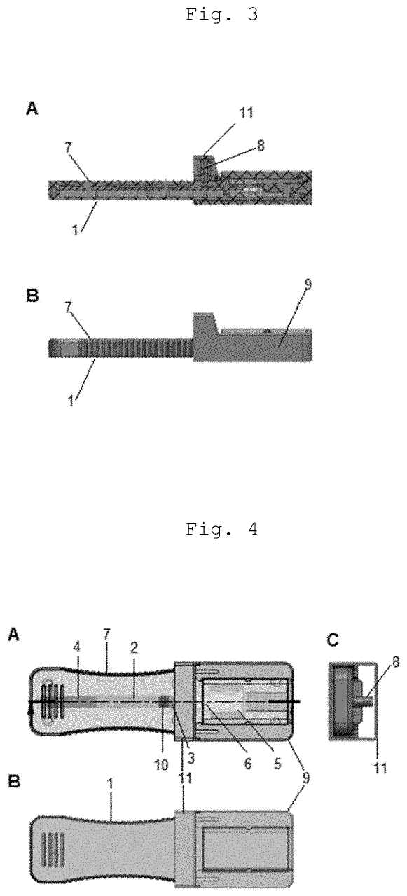

FIG. 3 shows a hollow side view (panel A) showing the interior of the device of one embodiment of the cassette device of the invention. The panel B shows a side view of the device of one embodiment of the cassette device of the invention.

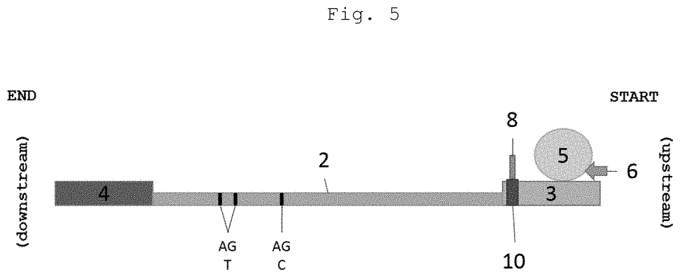

FIG. 4 shows a top view (panel A) and a bottom view (panel B) of one embodiment of the cassette device of the invention. The panel C shows a side view of the cassette device of the invention, especially the closure of the capillary-tube-shaped structure (8) promoted by the upper bar (11) of the movable cover (9).

FIG. 5 is a representative scheme of the internal structures of the device and its spatial arrangement therein.

### DETAILED DESCRIPTION OF THE INVENTION

The following description is provided to enable those skilled in the art to make and use the described embodiments contemplated for carrying out the invention. Various modifications, equivalents, variations and alternatives, however, will remain readily apparent to those skilled in the art. Any and all such modifications, variations, equivalents and alternatives should be within the spirit and scope of the present invention.

For purposes of the following description it is necessary to define some terms used throughout this description. For example, a “biological sample” is meant any sample taken from a patient for purposes of verifying the presence of antibodies through the device of the invention. For example, a biological sample may be capillary blood, venous blood, tissue fluids or a mixture thereof.

By “spear” or “spear-shaped structure” is meant any piercing-cutting device or structure responsible for disrupting or piercing a structure containing the buffer solution.

By “buffer solution” is meant any solution capable of maintaining the pH and/or the ideal conditions for running immunoassays. The buffer solution may be any buffer solution known in the art. As non-limiting examples of buffer solutions we can mention, Tris (tris (hydroxymethyl) aminomethane) buffer, Tris-glycine buffer, Tris-HCl (hydrochloric acid) buffer, citrate buffer and phosphate buffer. The buffer of choice will depend on the analyte or antibody to be detected in the biological sample, since each buffer acts in specific pH ranges.

By “immunoassay” or “immunochromatography” is meant a biochemical test that measures the presence of a small molecule in solution through the use of an antibody or an antigen, also referred to as an analyte.

By “antibody” is meant a biological molecule, such as glycoproteins or immunoglobulins, produced by cells of the immune system in response to the presence of pathogens in the blood. In the present description, “antibody” refers to a molecule present in the biological sample to be detected.

The terms “ligand” or “antigen”, in the context of the present invention, are interchangeable, and in that description they refer to a molecule similar to the pathogen to be detected, being complementary to the antibody produced by the immune system of a subject. Therefore, the antigen fixed to the test strip (2) of the invention will be complexed or bonded to an antibody present in the biological sample.

When the term “downstream” is mentioned throughout this description, it is understood to refer to the downstream or downstream side, i.e. a point posterior to the reference point. Similarly, the term “upstream” should be understood as a reference to the highest point, i.e. a point anterior to the reference point.

When reference is made herein to “control”, “reaction control”, “positive reaction control”, it is to be understood by the person skilled in the art as the presence of a ligand for a putative antibody present in the blood sample being tested. The presence of the positive control indicates whether the reaction of the rapid test device is positive, negative or invalid (in the case of non-appearance of the control line, which would indicate failure of the test). Its function, therefore, is to verify that the reagents of the rapid test device of the invention function satisfactorily.

In one embodiment, the invention in screen discloses a device for rapid diagnostic test of biological samples, preferably capillary blood, or a fluid mixture composed of blood from arterioles, venules and capillaries, as well as tissue fluid, wherein the sample is collected by any means available in the art, and the biological sample is inserted into the capillary-shaped structure (8) by microfluidics, by using the capillary force itself. Once the sample volume occupies the entire capillary-shaped structure (8) and reaches the adjacent absorbent pad (3), located at a posterior (or downstream) position of the capsule (5) containing the buffer solution, the entire volume of the sample is transferred to the absorbent pad (3) by capillarity, completely and immediately emptying the capillary-shaped structure (8). In this way, the insertion of excessive amounts of the volume of the biological sample to be analyzed is prevented, thus avoiding altered results of the diagnostic test by too much or insufficient sample.

The analytical procedure of the device on screen is based on the deposition of the sample on the first absorbent pad (3), as discussed above, which is mixed with a buffer solution for a more fluid test. The sample will migrate by capillarity to the second absorbent pad (4), passing through the test strip (2) having one or more of an antigen fixed thereto. Passage of the sample through this capture region will promote specific binding of the antibody to the antigen.

The efficiency of the test will be determined by the concentration of the reagents that promote the transport of the sample through the test strip (2), causing the sample to reach the test region.

Once the sample is collected and transferred to the first absorbent pad (3), for the reaction to start, the movable cover (9) is pressed into the closed position (FIG. 2, Panel A). Closing the movable cover (9) promotes the closure of the capillary-shaped structure (8) and the isolation of the interior of the device, while pressing the spear-shaped structure (6) on the capsule containing the buffer solution (5) promoting the disruption thereof. The capsule (5), when disrupted, promotes controlled pouring of the buffer solution contained therein onto the absorbent pad (3) in an anterior position (or upstream) the point or region receiving the blood sample (10) from the capillary-tube-shaped structure (8).

Controlled pouring of the buffer solution contained within the capsule (5) onto the absorbent pad (3) ensures that the sample meets all the conditions required for the desired reaction in the required volume, preventing waste, dilution of the sample, and washing of the system. Additionally and advantageously, the inlet position of the buffer solution in the system (in the anterior position in the absorbent pad (3) with respect to the sample inlet point (10)) allows for incubation of the biological sample with the buffer, providing the conditions required to the reaction and promoting the carrying of the entire biological sample in the reaction system. Uncontrolled disruption of the capsule (5) containing the buffer may cause the solution to be compelled in all directions, which may result in the sample being driven in a direction other than that of the reaction or even out of the device, and the same may happen with the buffer solution. The controlled pouring of the buffer solution prevents these and other certain disadvantages of the state of art presented in the previous section.

Once the reaction is started by compression of the movable cover (9) the buffer solution will go through the entire test strip (2) until it reaches the absorbent pad (4) in the posterior (upstream) portion of the device. Any biological sample deposited on the absorbent pad (10) will be led to go through the entire test strip (2) along with the buffer solution. During passage through the test strip (2), any antibody molecule present in the biological sample, if any, pushed upstream, when finding the antigen molecule or ligand fixed to the test strip (2), will be complexed thereto, in order to make a reversible or irreversible complex of the antibody molecule with the antigen molecule. Formation of this complex will be detected by observing by the naked eye the formation of the antibody-antigen complex formed within the rapid test device of the invention.

The formation of complex may alternatively be determined by any means known in the art.

It will be understood by one skilled in the art that for the determination of the efficiency of the rapid test device of the invention, and the presence of a ligand in a biological sample, it is necessary to detect a control molecule, such as a positive control, or negative control, confirming the operation of the system. The (positive and/or negative) control is given by the detection of any known molecule present in the biological sample.

Having disclosed some preferred and alternative embodiments of the present invention, it is clear that none of the examples provided is limiting to the scope of protection of the present invention, which is defined and limited solely by the appended set of claims and the claims therein.

1. A device for rapid diagnostic test containing a lower cassette containing recesses where a test strip containing one or more of a sample ligand and a positive control ligand of reaction characterized for comprising:
a lower cassette (1) containing recesses where a test strip (2) containing one or more of a sample ligand and a positive control ligand of reaction is positioned; a first absorbing pad (3) positioned over the test strip, upstream thereof; a second absorbing pad (4) positioned over the test strip (2), downstream thereof; a capsule (5) containing buffer upstream the test strip (2), and a spear-shaped structure (6) located in a position anterior the capsule (5),
an upper cassette (7) having a capillary-tube-shaped structure (8) to be filled with a blood sample, located over the absorbing pad (3) upstream, and that is fitted to the lower cassette (1) forming an assembly containing an inner cavity isolated from external environment, where the rapid test reaction occurs,
a compressible, movable cover (9) that is connected to the lower cassette (1) and to the upper cassette (7) upstream the capillary-shaped structure (8), promoting assembly closure, wherein compressing the movable cover (9) causes the spear-shaped structure (6) to press the capsule (5) containing buffer, such that said capsule (5) is disrupted, pouring the buffer solution over the absorbing pad (3) located upstream the test strip (2), going through entire test strip (2) until it is absorbed by the absorbing pad (4) located downstream the test strip (2), and,
wherein compressing the movable cover (9) promotes capillary-tube-shaped structure closure (8).

2. The device, according to claim 1, characterized by the fact capsule disruption (5) containing buffer occurs by compressing the movable cover (9) over the spear-shaped structure (6), causing the buffer contained in the capsule (5) to be poured in a controlled manner over the absorbing pad (3) located upstream the test strip (2), in the region anterior the region receiving the blood sample (10) from the capillary-tube-shaped structure (8).

3. The device, according to claim 1, characterized by the fact that buffer displacing in the absorbing pad (3) and over the test strip (2) pushes the components of the blood sample throughout the test strip (2) such that the assembly formed by the buffer and the components of the blood goes through the entire test strip (2), passing through the regions containing a sample ligand and a positive control ligand of reaction, until it achieves the absorbing pad (4) downstream the test strip (2).

4. The device, according to claim 1, characterized in that the capillary-tube-shaped structure (8) is located over the absorbing pad (3), and in direct contact with the absorbing pad (3) in a position (10) posterior the capsule containing buffer (5) and anterior the portion of the test strip (2) containing a sample ligand and a positive control ligand of reaction.

5. The device, according to claim 1, characterized in that the blood sample is inserted to the capillary-tube-shaped structure (8), by microfluidics, such that, after being completely filled, the sample achieves the absorbing pad (3), and is immediately transferred thereto, causing the capillary-tube-shaped structure (8) to be emptied.

6. The device, according to claim, characterized in that the capillary-tube-shaped structure (8) is able to hold the needed sample amount to run the rapid test of the device, as defined in claim 1.

7. The device, according to claim 1, characterized in that after transferring the blood sample of the capillary-tube-shaped structure (8) to the absorbing pad (3) the capillary-tube-shaped structure (8) emptying occurs, preventing transferring additional blood sample volume.

8. The device, according to claim 1, characterized in that the test strip is a nitrocellulose membrane strip.

9. The device, according to claim 1, characterized in that the lower cassette (1) and upper cassette (7) bodies of the device have a transparent material, allowing to see the blood sample and the buffer inside the device, from the sample entry to its absorption by the absorbing pad (4).

10. The device, according to claim 1 characterized in that the movable cover (9) has an upper cross bar (11) that, when the movable cover (9) is compressed, it is located over the capillary-tube-shaped structure (8) causing the capillary-tube-shaped structure closure (8) and the isolation of the inner portion of the device from external environment.

11. The device, according to claim 1, characterized in that a ligand of a sample located in the test strip (2) corresponds to one or more antigens fixed to the test strip (2) in a portion posterior the region receiving the blood sample (10) from the capillary-tube-shaped structure (8).

12. The device, according to claim 11, characterized in that one or more antigens present in the test strip (2) are complementary to one or more antibodies present in the capillary blood sample.

13. A method for identifying one or more antigens in a biological sample characterized for having the following steps:
collecting a biological sample, as a capillary blood sample;
filling the capillary-tube-shaped structure (8) of the device, as defined in claim 1 in its entirety with the needed amount of the blood sample collected;
pressing the movable cover (9) of the device, as defined in the claim 1, so as to cause capillary-tube-shaped structure closure (8) and capsule compression (5) containing buffer solution, that is poured upstream the biological sample;
waiting for the buffer solution to go through entire test strip (2) until it is absorbed by the absorbing pad positioned over the test strip (4), and downstream thereof; and
verifying the formation of one or more conjugates among the one or more antigens present in the test strip (2), and one or more antibodies present in the biological sample.

14. The method, according to claim 13, characterized in that the biological sample is capillary blood, venous blood, tissue fluid or a mixture thereof.

15. The method, according to claim 13, characterized for being run with the device as defined in claim 12.

16. A kit of rapid diagnostic test characterized for comprising:
the device as defined in claim 1; and
instructions for use.
